(19) United States
(12) Patent Application Publication
(10) Pub. No.: US 2005/0273870 A1
(43) Pub. Date: Dec. 8, 2005

(54) Preparation and selection of donor cells for nuclear transplantation

(75) Inventors: James Robl, Beichertown, MA (US); Kasinathan Poothappillai, Sioux Falls, SD (US); Jason G. Knott, Lancaster, MA (US); Joseph D. Jerry, Shutasbury, MA (US)

Correspondence Address: MERCHANT & GOULD PC, P.O. BOX 2903, MINNEAPOLIS, MN, 55402-0903, US

(73) Assignee: University of Massachusetts

(21) Appl. No.: 11/174,825

(22) Filed: Jul. 5, 2005

Related U.S. Application Data
(63) Continuation of application No. PCT/US00/28287, filed on Oct. 13, 2000.
(60) Provisional application No. 60/159,368, filed on Oct. 14, 1999.

(51) Int. Cl. A01K 67/00; C12N 15/85

(52) U.S. Cl. 800/8; 800/21; 435/455

(57) ABSTRACT

The invention relates to a method of synchronizing a population of somatic cells in G₁ for purposes of preparing the cells for nuclear transfer or nuclear transplantation by using mechanical shake-off and selection of mitotic doublet cells. This method may further comprise cooling of the cells or other means of synchronizing the cells in G₁ phase for longer periods of time. The invention also relates to the use of a synchronized population of rapidly, dividing somatic cells obtained by these methods as a source of donor nuclei or chromatin for use in nuclear transfer or nuclear transplantation.

### BACKGROUND OF THE INVENTION

A. Cell Synchrony

An important tool for cell cycle analysis is the ability to place cells in the same phase of cell cycle (e.g., S, M, G₁ or G₂). Cell synchronization has been performed for years and can be performed with or without the aid of chemicals. One of the best methods of synchronization uses the fact that spherical, mitotic (M) phase cells adhere less firmly to glass surfaces than do interphase cells (e.g., interphase cells are those cells in S, G₁ or G₂). Therefore, by shaking the cell cultures one can isolate large numbers of uncontaminated M phase cells (see JAMES D. WATSON ET AL., MOLECULAR BIOLOGY OF THE GENE 971 (4^{th} ed., 1987). The non-chemical technique of “shake-off” works well with Chinese hamster ovary (CHO) cells and some sublines of HeLa (R. IAN FRESHNEY, CULTURE OF ANIMAL CELLS: A MANUAL OF BASIC TECHNIQUES 384-385 (3^{rd} ed. 1994); and Zwanenburg, Mutat. Res. 120: 151-9 (1983)). Success comparable to that observed with CHOs has been achieved in synchronizing diploid human fibroblasts using mechanical shake-off (Tobey et al., Exp. Cell Res. 179: 400-16 (1988)). Shake-off has also been used to synchronize embryonic quail skeletal myoblasts (Devlin et aL, Dev. Biol. 95: 175-92 (1983)) and HeLa cells (Wheatley et al, Cytobios. 55: 191-204 (1988)). Another mechanical means of synchronizing cells in G₁ is to use centrifugal elutriation, which can cause the cells temporarily arrest in G₀ (Zickert et al,. Exp. Cell. Res. 207:115-21 (1993)).

Cell synchronization can also be achieved by using a combination of mechanical shake-off and chemicals (e.g., aphidicolin) (Graves et al., Anal. Biochem. 248: 251-7 (1997)). However, use of drugs (e.g., aphidicolin or hydroxyurea) have toxic side effects on CHOs, whereas shake-off does not (Fox et al., Cytometry 8: 3 15-20 (1987)). Drugs can also be used alone to synchronize cells. G₁ and/or G₀ arresting drugs include dexamethasone (Goya et al., Mol. Endocrinol. 7: 1121-32 (1993)), as well as other glucocorticoids (Sanchez et al., Cell Growth Differ. 4: 2 15-25 (1993)), or bidentate 3-hydroxypyridin-4-one (HPO) and hexdentate desferrioxamine (DFO) (Hoyes et al., Cancer Res. 52: 4591-9 (1992)). Other G₁-specific cell cycle synchronizing agents are discussed in Gadbois et al., Proc. Nat'l Acad. Sci. USA 89: 8626-8630 (1992).

Temperature has also been employed to mediate the cell cycle of a cell. Cold-shock synchronizes immature granulocytic cells from peripheral blood or bone marrow (Boucher et al., Hum. Genet. 54: 207-11 (1980)). Human diploid fibroblasts are arrested in G₁ by switching the cells to low temperature, such as 30° C. (Enninga et al., Mutat. Res. 130: 343-52 (1984)). Temperature was used to stop cell cycle in G₁, S, late S and G₂+M phases after the CHO cells were synchronized using the mechanical shake-off procedure (Schneiderman et al., Radiat. Res. 116: 283-91 (1988)).

The cell cycle stage of donor cell nuclei critically affects the chromatin structure and development of nuclear transplant embryos. Synchronization of the donor nucleus in the G₁ phase is an important factor for successful development of nuclear transplant embryos (Cheong et al., Biol. Reprod. 48: 958-63 (1993)). Specifically, late S chromatin influences chromosome constitution in embryos and may account for the reduced development of nuclear transplant embryos when late S phase donor nuclei are used (Collas et al., Biol. Reprod. 46: 501-11 (1992)). Cell cycle influences the use of a donor nucleus on chromatin structure and development of mouse embryonic nuclei transplanted into enucleated oocytes. Cell-cycle synchronization has also been shown to play an important role in the use of porcine ectodermal cell donor nuclei in nuclear reprogramming of the nuclei material after the donor is fused to an enucleated metaphase-II oocyte (Ouhibi et al., Mol. Reprod. Dev. 44: 533-9 (1996)). However, cell synchronization, for purposes of nuclear transplantation of somatic cell nuclei, has not used mitotic cell shake-off in combination with doublet cell selection. Moreover, the shake-off and doublet selection of somatic cells has not been used in combination with other methods of cell cycle synchronization (e.g., G₁ phase arresting agents or methods) for the purpose of preparing somatic cell nuclei for transplantation.

B. Preparing Somatic Cells for Nuclear Transplantation or Nuclear Transfer

In 1996, the first successful transfer of a nucleus from an adult mammary gland cell into an enucleated oocyte was reported (Campbell et al., Nature 380: 64-6 (1996)). This success was followed by the production of rhesus monkeys by nuclear transfer of embryonic cells. Nuclear transfer involves preparing a cytoplast as a recipient cell. In most cases, the cytoplast is derived from a mature metaphase II oocyte from which the chromosomes have been removed. A donor cell nucleus is then placed between the zona and the cytoplast; fusion, as well as cytoplast activation, is initiated by electrical stimulation. Successful reprogramming of the donor cell nucleus by the cytoplast is critical, and is a step which may be influenced by cell cycle. See Wolf et al., Biol. Reprod. 60: 199-204 (1999). A number of pregnancies have been established using fetal cells as the source of donor nuclei. However, the use of cell lines to create transgenic animals permits large clone sizes and the genetic manipulation of the cells in vitro before nuclear transfer. Id. The mechanisms regulating early embryonic development may be conserved among mammalian species, such that, for example, bovine oocyte cytoplasm can support the introduced differentiated donor nucleus regardless of chromosome number, species or age of the donor fibroblast (Dominko et al., Biol. Reprod. 60:1496-502 (1999)).

Actively dividing fetal fibroblasts can be used as nuclear donors according to the procedure described in Cibelli et al., Science 280: 1256-9 (1998). Additional methods of preparing recipient oocytes for nuclear transfer of donor differentiated nuclei are as described in International PCT Application Nos. 99/05266; 99/01164; 99/01163; 98/3916; 98/30683; 97/41209; 97/07668; and U.S. Pat. No. 5,843,754. Typically the transplanted nuclei are from cultured embryonic stem (ES), embryonic germ (EG) cells or other embryonic cells See International PCT Applications Nos. 95/17500 and 95/10599; Canadian Patent No. 2,092,258; Great Britain Patent No. 2,265,909; and U.S. Pat. Nos. 5,453,366; 5,057,420; 4,994,384; and 4,664,097. Inner cell mass (ICM) cells can also be used as nuclear donors (Sims et al., Proc. Natl Acad. Sci. USA 90: 6143-6147 (1990); and Keefer et al., Biol. Reprod. 50: 935-939 (1994)).

C. Transgenic Animals and Production Thereof

Pronuclear Microinjection. Various methods have been utilized in an attempt to genetically modify animals so as to introduce superior qualities including pronuclear microinjection. One of the limitations of pronuclear microinjection is that the gene insertion site is random. This typically results in variation of expression levels, and several transgenic lines must be produced to obtain one line with an appropriate level of expression. Because integration is random, it is advantageous that lines of transgenic animals are started from one founder animal to avoid difficulties in monitoring zygosity and potential difficulties that might occur with interactions among multiple insertion sites (Cundiff et al., J. Animal Sci. 71: 20-25 (1993)). Even without concern for inbreeding, it would take about 6.5 years before reproduction could be tested in homozygous animals (Seidel, J. Animal Sci. 71: 26-33 (1993)).

A second limitation of the pronuclear microinjection procedure is its efficiency. Only 0.34 to 2.63% of the gene-injected embryos develop into transgenic animals, and a fraction of these appropriately express the gene (Purcel et al., J. Animal Sci 71:10-19 (1993)). This inefficiency results in a high cost of producing transgenic animals because of the large number of recipients required. Thus, the ability to clone, or to make numerous identical genetic copies, of an animal comprising a desired genetic modification would be advantageous.

Embryonic Stem Cells. Another system for producing transgenic animals has been developed that uses embryonic stem (ES) cells. In mice, ES cells have enabled researchers to select for transgenic cells and perform gene targeting. This method allows more genetic engineering than is possible with other transgenic techniques. For example, ES cells are relatively easy to grow as colonies in vitro, can be transfected by standard procedures, and the transgenic cells clonally selected by antibiotic resistance (T. Doetschman, “Gene transfer in embryonic stem cells.” IN TRANSGENIC ANIMAL TECHNOLOGY: A LABORATORY HANDBOOK 115-146 (C. Pinkert, ed., Academic Press, Inc., N.Y. 1994)). Furthermore, the efficiency of this process is such that sufficient transgenic colonies (hundreds to thousands) can be produced to allow a second selection for homologous recombinants. Id. ES cells can then be combined with a normal host embryo and, because they retain their potency, and can develop into all the tissues in the resulting chimeric animal, including the germ cells. Therefore, transgenic modification is transmissible to subsequent generations.

Methods for deriving embryonic stem (ES) cell lines in vitro from early preimplantation mouse embryos are well known (Evans et al., Nature 29: 154-156 (1981); Martin, Proc. Natl. Acad. Sci. USA 78: 7634-7638 (1981)). ES cells can be passaged in an undifferentiated state, provided that a feeder layer of fibroblast cells (Evans et al., 1981) or a differentiation inhibiting source (Smith et al., Dev. Biol. 121:1-9 (1987)) is present.

In view of their ability to transfer their genome to the next generation, ES cells have potential utility for germline manipulation of livestock animals. Some research groups have reported the isolation of purportedly pluripotent embryonic cell lines. For example, Notarianni et al., J. Reprod. Fert. Suppl. 43: 55-260 (1991) reported the establishment of stable, pluripotent cell lines from pig and sheep blastocysts, which exhibit some morphological and growth characteristics similar to that of cells in primary cultures of inner cell masses (ICMs) isolated immunosurgically from sheep blastocysts. Also, Notarianni et al., J. Reprod. Fert. Suppl. 41: 51-56 (1990) disclosed maintenance and differentiation in culture of putative pluripotent embryonic cell lines from pig blastocysts. Gerfen et al., Anim. Biotech. 6:1-14 (1995) disclosed the isolation of embryonic cell lines from porcine blastocysts. These cells are stably maintained without mouse embryonic fibroblast feeder layers and reportedly differentiate into several different cell types during culture.

Further, Saito et al., Roux's Arch. Dev. Bid. 201: 134-141 (1992) reported cultured, bovine embryonic stem cell-like cell lines, which survived three passages, but were lost after the fourth passage. Handyside et al, Roux's Arch. Dev. Biol. 196:185-190 (1987) disclosed culturing immunosurgically isolated inner cell masses (ICMs) of sheep embryos under conditions that allow for the isolation of mouse ES cell lines derived from mouse ICMs.

Chemy et al., Theriogenology 41:175 (1994) reported purportedly pluripotent bovine primordial germ cell-derived cell lines maintained in long-term culture. These cells, after approximately seven days in culture, produced ES-like colonies, which stained positive for alkaline phosphatase (AP), exhibited the ability to form embryoid bodies, and spontaneously differentiated into at least two different cell types.

Campbell et al., (1996) reported the production of live lambs following nuclear transfer of cultured embryonic disc (ED) cells from day nine ovine embryos cultured under conditions which promote the isolation of ES cell lines in the mouse.

Van Stekelenburg-Hamers et al., Mol. Reprod. Dev. 40: 444-454 (1995) reported the isolation and characterization of purportedly permanent cell lines from ICMs of bovine blastocysts. The authors isolated and cultured ICMs from eight or nine day bovine blastocysts under different conditions to determine which feeder cells and culture media are most efficient in supporting the attachment and outgrowth of bovine ICM cells.

Purportedly, animal stem cells have been isolated, selected and propagated for use in obtaining transgenic animals (see Evans et al., WO 90/03432; Smith et al., WO 94/24274; and Wheeler et al., WO 94/26884). Evans et al. also reported the derivation of purportedly pluripotent ES cells from porcine and bovine species, which assertedly are useful for the production of transgenic animals.

ES cells from a transgenic embryo could be used in nuclear transplantation. The use of ungulate ICM-cells for nuclear transplantation-also has been reported. In the case of livestock animals, e.g., ungulates, nuclei from similar preimplantation livestock embryos support the development of enucleated oocytes to term (Keefer et al., 1994; Smith et al., Biol. Reprod. 40:1027-1035 (1989)). In contrast, nuclei from mouse embryos do not support development of enucleated oocytes beyond the eight-cell stage after transfer (Cheong et al., Biol. Reprod. 48: 958 (1993)). Therefore, ES cells from livestock animals are highly desirable, because they may provide a potential source of totipotent donor nuclei, genetically manipulated or otherwise, for nuclear transfer procedures.

Use of ICM Cells. Collas et al., Mol. Reprod. Dev. 38: 264-267 (1994) disclosed nuclear transplantation of bovine ICMs by microinjection of the lysed donor cells into enucleated mature oocytes. Culturing of embryos in vitro for seven days produced fifteen blastocysts which, upon transfer into bovine recipients, resulted in four pregnancies and two births. Also, Keefer eta?., Biol. Reprod. 50: 93 5-939 (1994) disclosed the use of bovine ICM cells as donor nuclei in nuclear transfer procedures, to produce blastocysts which also resulted in several live offspring. Further, Sims et al., Proc. Natl. Acad. Sci. USA 90: 6143-6147 (1993) disclosed the production of calves by transfer of nuclei from short-term in vitro cultured bovine ICM cells into enucleated mature oocytes.

Therefore, notwithstanding what has previously been reported in the literature, there exists a need for improved methods of preparing large numbers of cells for nuclear transfer or transplantation for use in creating transgenic or chimeric animals. Using cell cycle-synchronized cells, which represent a rapidly dividing sub-population, as donor nuclei will enhance the development of transgenic or chimeric animals, especially livestock animals, using nuclear transfer procedures.

### SUMMARY AND OBJECTS OF THE INVENTION

It is an object of the present invention to provide a method of preparing donor somatic cells for nuclear transfer or nuclear transplantation comprising the steps of: (A) synchronizing the cell cycle of donor somatic cells by shaking the cells; (B) selecting doublet cells from said shaken somatic cells; and (C) preparing the selected mitotic double cells for nuclear transfer. A cooling step can optionally be included wherein the selected doublet cells are cooled to below metabolic temperature in order to lengthen the G₁ phase. Also, optionally, the numbers of cells in G₁ phase or the period of G₁ can be enhanced by placing the cells in appropriate media, e.g., media lacking at least one of the following: serum, isoleucine, glutamine or phosphate or by the addition of a G₁ synchronizing agent (e.g., aphidicolin or mimosine).

It is a specific object of the invention to obtain cells which are rapidly dividing somatic cells, for example (cell cycle completion in 15 hours or less, more preferably 10 hours or less). Such cells can be obtained using the methods described above.

### BRIEF DESCRIPTION OF THE DRAWINGS

FIG. 1: Effect of Confluency and Cell Age on Cell Cycle Length. The histogram describes the difference in cell cycle lengths (measured in hours) of cells at 25% confluence versus cells at 90% confluency. Cell cycles were observed in cell populations obtained from 40 day old fetuses (40D FET), 4 year old cows (4 YRS), 15 year old cows (15 YRS) and total cells.

FIG. 2: Effect of Time in Culture and Donor Age on Cell Growth Rate. The cell growth rate was compared for cells derived from 40 day old fetuses (40D FET), calves aged from 0-13 months (0-13 MO) and calves aged 24-72 months (24-72). Population doubling (PD) is compared depending on the number of days the cells are in culture. The mean PD decreases as the number of days in culture increases.

FIG. 3: Length of G₁ in Fibroblasts Recovered from Culture.

### DETAILED DESCRIPTION OF THE INVENTION

For a better understanding of the invention, the following detailed description refers to the accompanying drawings and examples, wherein preferred exemplary embodiments of the present invention are illustrated and described. This invention relates to a novel method of obtaining somatic cells as donor nuclei, which provides a population of donor nuclei temporally optimized for nuclear transfer or nuclear transplantation.

A. Definitions

By “synchronized cells” or “synchronizing” is meant a culture of cells or a method of preparing said cells such that more than 90% of the cells are in G₁ phase.

By “confluent cells” is meant cell population densities of about 90% or greater.

The terms “nuclear transfer” or “nuclear transplantation” refer to a method of cloning, wherein the donor cell nucleus is transplanted into a cell cytoplast. The cytoplast could be from an enucleated oocyte, an enucleated ES cell, an enucleated EG cell, an enucleated embryonic cell or an enucleated somatic cell. Nuclear transfer techniques or nuclear transplantation techniques are known in the literature (Campbell et al., Theriogenology 43: 181 (1995); Collas et al., (1994); Keefer et al., (1994); Sims et al., (1993); Evans et al., WO 90/03432; Smith et al., WO 94/24274; and Wheeler et al., WO 94/26884. Also U.S. Pat. Nos. 4,994,384 and 5,057,420 describe procedures for bovine nuclear transplantation. In the subject application, “nuclear transfer” or “nuclear transplantation” or “NT” are used interchangeably.

The terms “nuclear transfer unit” and “NT unit” refer to the product of fusion between or injection of a somatic cell or cell nucleus and an enucleated cytoplast (e.g., an enucleated oocyte), which is sometimes referred to herein as a fused NT unit.

By “somatic cell” is meant any cell of a multicellular organism, preferably an animal, that does not become a gamete. The preferred “somatic cell ” are adherent cells. By “adherent cells” are meant cells that when cultured adhere to the surface of the tissue culture flask or other such compartment.

By “animal” is meant to include mammals, e.g. livestock animals (e.g., ungulates such as cattle, buffalo, horses, sheep, pigs and goats), as well as rodents(e.g., mice, hamsters, rats and guinea pigs), domesticated animals such as canines, felines horses, rabbits and primates. Animals also include endangered or even extinct species such as guar, giant pandas, elephants, a African bongo antelope, Sumatran tiger, bucardo mountain goat, cheetah, and ocelot, et seq.

By “doublet cell” is meant to include those cells which are attached by cytoplasmic bridges. A “cytoplasmic bridge” occurs during the final stages of cytokinesis, before the daughter cells complete separation.

By “rapidly dividing cell” is meant a cell being grown in a low population density (50% population density or less) in media containing serum.

By “G₁ synchronizing agent” is meant an agent which enhances the production of cells in, or arrests a cell in, G₁ .

By “chimera” or “chimeric animal” is meant an organism composed of two genetically distinct types of cells. The chimera can be formed by the fusion of two early blastula stage embryos, for example.

By “transgenic animal” is mean an organism that has integrated into its genome one or more foreign DNA molecules.

B. Cell Cycle Synchronization

Somatic cell synchronization will be performed using mitotic shake-off wherein cells are shaken by slapping tissue culture flasks to knock-off mitotic cells from the flask wall. In brief 0.5×10⁶ cells are plated 24 hours prior to shake-off. Shake-off is carried out typically by placing the flask or other tissue culture dish on a vortexer or other shaking apparatus are for about 30 to about 60 seconds. Media containing the cells which are shaken off is removed and centrifuged. The pelleted cells are resuspended in 250 ,μ1 of medium. Doublet cells are separated from non-doublet cells obtained at the shake-off step by visual inspection. Doublet cells can also be isolated, e.g., by centrifugation using a gradient to separate doublet cells from non-doublet cells.

These cells can immediately be used for nuclei removal for nuclear transplantation or nuclear transfer. Alternatively, the cells can be cooled to below metabolic temperature (e.g., below 37° C., more preferably 4-20° C., and most preferred at 4° C.) to maintain the period that they are in G₁. The cells can also be kept in G₁ phase using other means, such as serum deprivation or depletion of isoleucine, glutamine or phosphate from the media after doublet selection. Drugs, such as colchicine, blocks cells in M phase (JAMES D. WATSON ET AL., MOLECULAR BIOLOGY OF THE GENE 973 (4^{th} ed., 1987). Other drugs can block cells in G₁ phase, such as mimosine (Krude, Exp. Cell. Res. 247:148-59 (1999)), glucocorticoids (Sanchez et al., Cell Growth Differ. 4: 2 15-25 (1993)) aphidicolin and certain kinase inhibitors (e.g., KT5720, KT5823, KT5926 and K5256 described in Gadbois et al., 1992). Other drugs block cells at the G₁-S border, including bidentate 3-hydroxypyridin-4-one iron chelators and hexadentate desferrixoxamine (Hoyes et al., Cancer Res. 52: 459 1-9 (1992)). These drugs can be added to the media of the selected doublet cells to lengthen the period of the G₁ phase.

C. Nuclear Transplantation and Development of Transgenic Animals Using Somatic Cells

The use of adult cells and fetal fibroblast cells from a sheep have been used as nuclear transfer donors to produce a cloned sheep offspring (Wilmut et al., Nature 385: 810-813 (1997)). However, in that study, it was emphasized that the use of a serum starved, nucleus donor cell in the quiescent state was important for success of the Wilmut cloning method. No such requirement for serum starvation or quiescence for maintaining the cells in G₀ exists for the present invention. On the contrary, cloning is achieved using differentiated mammalian cells proceeding through cell cycle, i.e., cells in G₁, G₂ or M or S phases.

Thus, in one aspect, the present invention provides an improved method for cloning an animal. In general, the animal will be p Lourenco roduced by a nuclear transfer process comprising the following steps:

(i) obtaining desired somatic cells by the methods described herein, which may be serum or non-serum starved, to be used as a source of donor nuclei;

(ii) obtaining oocytes from an animal, e.g., bovine;

(iii) enucleating said oocytes;

(iv) transferring the desired somatic cell or cell nucleus into the enucleated oocyte, e.g., by fusion or injection, to form an NT unit;

(v) (v) activating the NT unit to yield an activated NT unit; and

(vi) (vi) transferring said activated NT unit to a host animal such that the NT unit develops into a fetus.

Optionally, the activated NT unit is cultured beyond the 2-cell developmental stage prior to transfer to the host animal.

The present invention also includes a method of cloning a genetically engineered or transgenic animal, by which a desired DNA sequence is inserted, removed or modified in the serum or non-serum starved differentiated animal cell or cell nucleus prior to insertion of the differentiated animal cell (e.g., somatic cell) or cell nucleus into an oocyte, which is enucleated before or after nuclear transfer.

In addition to the uses described above, the genetically engineered or transgenic animals according to the invention can be used to produce a desired protein, such as a pharmacologically important protein, e.g., human serum albumin. That desired protein can then be isolated from milk or other fluids or tissues of the transgenic animal. Alternatively, the exogenous DNA sequence may confer an agriculturally useful trait to the transgenic animal, such as disease resistance, decreased body fat, increased lean meat product, improved feed conversion, or altered sex ratios in progeny.

The stage of oocyte maturation at enucleation and nuclear transfer has been reported to be significant to the success of NT methods (Prather et al., Differentiation 48: 1-8 (1991)). In general, successful mammalian embryo cloning practices use a metaphase II stage oocyte as the recipient oocyte, because at this stage it is believed that the oocyte can reprogram the nucleus by causing disassembly of the nucleus and condensation of the chromatin. Activation of the NT unit is then induced. In domestic animals, the oocyte activation period generally ranges from about 16-52 hours, preferably about 20-45 hours post-aspiration.

Methods for isolating of oocytes are well known in the art. Essentially, this comprises isolating oocytes from the ovaries or reproductive tract of an animal, e.g., a bovine. A readily available source of bovine oocytes is from slaughterhouse materials.

For the successful use of techniques such as genetic engineering, nuclear transfer and cloning, oocytes must generally be matured in vitro before these cells may be used as recipient cells for nuclear transfer, and before they can be fertilized by the sperm cell to develop into an embryo. This process generally requires collecting immature (prophase I) oocytes from mammalian ovaries, e.g., bovine ovaries obtained from a slaughterhouse, and maturing the oocytes in a maturation medium prior to fertilization or enucleation until the oocyte attains the metapbase II stage, which in the case of bovine oocytes generally occurs about 18-24 hours post-aspiration. For purposes of the present invention, this period of time is known as the “maturation period.” As used herein for calculating time periods, “aspiration” refers to aspiration of the immature oocyte from ovarian follicles.

Additionally, metaphase II stage oocytes, which have been matured in vivo, have been successfully used in nuclear transfer techniques. Essentially, mature, cow metaphase II oocytes can be collected surgically from either non-superovulated or superovulated cows or heifers from about 20 to about 30 hours past the onset of estrus or past the injection of human chorionic gonadotropin (hCG) or similar hormone.

The stage of maturation of the oocyte at enucleation and nuclear transfer has been reported to be significant to the success of NT methods. (See, Prather et al., Differentiation 48: 1-8 (1991)). In general, successful mammalian embryo cloning practices use the metaphase II stage oocyte as the recipient oocyte, because at this stage it is believed that the oocyte can be or is sufficiently “activated” to treat the introduced nucleus as it does a fertilizing sperm. In domestic animals, and especially cattle, the oocyte activation period generally ranges from about 16-52 hours, preferably about 28-42 hours post-aspiration.

For example, immature oocytes may be washed in HEPES buffered hamster embryo culture medium (HECM), as described in Seshagine et al., Biol. Reprod., 40:544-606 (1989), and then placed into drops of maturation medium consisting of 50 μ1 of tissue culture medium (TCM) 199 containing 10% fetal calf serum, which further contains appropriate gonadotropins such as luteinizing hormone (LH) and follicle stimulating hormone (FSH), and estradiol under a layer of lightweight paraffin or silicon at 39° C.

After a fixed time maturation period, which ranges from about 10 to about 40 hours, and preferably about 16-18 hours, the oocytes will be enucleated. Prior to enucleation the oocytes will preferably be removed and placed in HECM containing 1 mg/ml of hyaluronidase prior to removal of cumulus cells. This may be effected by either repeated pipetting through very fine bore pipettes or by vortexing briefly. The stripped oocytes are then screened for polar bodies, and the selected metaphase II oocytes, as determined by the presence of polar bodies, are then used for nuclear transfer.

Method of Enucleating Cells. Enucleation may be effected by known methods, such as described in U.S. Pat. No. 4,994,384, which is incorporated by reference herein. For example, metaphase II oocytes are either placed in HECM, optionally containing 7.5 μg/ml cytochalasin B, for immediate enucleation, or may be placed in a suitable medium, for example an embryo culture medium, such as CR1aa (CR1 media is described in U.S. Pat. No. 5,096,822. CR1aa is supplemented with amino acids), plus 10% estrus cow serum, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 μg/ml 33342 Hoechst dye in HECM, and then viewing the oocytes under ultraviolet irradiation for less than 10 seconds. Oocytes successfully enucleated can then be placed in a suitable culture medium, e.g., CR1aa supplemented with 10% serum.

In the present invention, the recipient oocytes will preferably be enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation.

A single mammalian somatic cell of the same species or different species will then be transferred into the perivitelline space of the oocyte used to produce the NT unit. Very recently, it was reported that to transfer of guar cells into an enucleated bovine oocyte resulted in a viable embryo (Scientific American Lonza et al., Oct. 2000). The mammalian cell and the oocyte will be used to produce NT units according to methods known in the art. For example, the cells may be fused by electrofusion. Electrofusion is accomplished by providing a pulse of electricity sufficient to cause a transient breakdown of the plasma membrane. This breakdown of the plasma membrane is very short-lived, because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated herein by reference in its entirety) for a further discussion of this process. A variety of electrofusion media can be used including, e.g., sucrose, mannitol, sorbitol and phosphate buffered solution. Fusion can also be accomplished using Senclai virus as a fusogenic agent (Graham, Wistar Inst. Symp. Monogr. 9:19 (1969)).

Also, in some cases (e.g., with small donor nuclei) it may be preferable to inject the nucleus directly into the oocyte rather than using electroporation fusion. Such techniques are disclosed in Collas et al., Mol. Reprod. Dev., 38: 264-267 (1994), incorporated by reference in its entirety herein.

Preferably, the somatic or germ cell and oocyte are electrofused in a 500 p.m chamber by application of an electrical pulse of about 90-120 V for about 15 μsec, about 24 hours after initiation of oocyte maturation. After fusion, the resultant fused NT units are then placed in a suitable medium until activation, e.g., CR1aa medium. Typically activation will be effected shortly thereafter, typically less than 24 hours later, and preferably about 4-9 hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may be affected by simultaneously or sequentially:

(i) increasing levels of divalent cations in the oocyte, and

(ii) reducing phosphorylation of cellular proteins in the oocyte. This will generally be effected by introducing divalent cations into the oocyte cytoplasm, e.g., magnesium, strontium, barium or calcium, e.g., m the form of an ionophore. Other methods of increasing divalent cation levels include the use of electric shock, treatment with ethanol and treatment with caged chelators.

Phosphorylation may be reduced by known methods, e.g., by the addition of kinase inhibitors, (e.g., serine-threonine kinase inhibitors, such as 6-dimethyl-aminopurine, staurosporine, 2-aminopurine, and sphingosine).

Alternatively, phosphorylation of cellular proteins may be inhibited by introduction of a phosphatase into the oocyte, e.g. phosphatase 2A and phosphatase 2B.

In one embodiment, NT activation is effected by briefly exposing the fused NT unit to a TL-HEPES medium containing 5 μM ionomycin and 1 mg/ml BSA, followed by washing in TL-HEPES containing 30 mg/ml BSA within about 24 hours after fusion, and preferably about 4 to about 9 hours after fusion.

The activated NT units may then be cultured in a suitable in vitro culture medium until the generation of cultured inner cell mass (CICM) cells and cell colonies. Culture media suitable for culturing and maturation of embryos are well known in the art. Examples of known media, which may be used for bovine embryo culture and maintenance, include Ham's F-10+10% fetal calf serum (FCS), Tissue Culture Medium-199 (TCM-199) supplemented with 10% fetal calf serum, Tyrodes-Albumin-Lactate-Pyruvate (TALP), Dulbecco's Phosphate Buffered Saline (PBS), Eagle's and Whitten's media. A common media used for the collection and maturation of oocytes is TCM-199, supplemented with 1 to 20% FCS, newborn serum, estrual cow serum, lamb serum or steer serum. A preferred maintenance medium includes TCM-199 with Earl salts, 10% fetal calf serum, 0.2 mM Na pyruvate and 50 μg/ml gentamicin sulphate. Any of the above may also involve co-culture with a variety of cell types, such as granulosa cells, oviduct cells, BRL cells and uterine cells and STO cells.

Another maintenance medium is described in U.S. Pat. No. 5,096,822 to Rosenkrans, which is incorporated herein by reference. This embryo medium, named CR1, contains the nutritional substances necessary to support an embryo.

For example, the activated NT units may be transferred to CR1aa culture medium containing 2.0 mM DMAP (Sigma) and cultured under ambient conditions, e.g., about 38.5° C., 5% CO₂ for a suitable time, e.g., about 4 to about 5 hours.

Afterward, the cultured NT unit or units are preferably washed and then placed in a suitable media, e.g., CR 1aa medium containing 10% FCS and 6 mg/ml contained 20 in well plates, which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

The methods for embryo transfer and recipient animal management in the present invention are standard procedures used in the embryo transfer industry. Synchronous transfers are important for success of the present invention, ie., the stage of the NT embryo is in synchrony with the estrus cycle of the recipient female. This advantage and how to maintain recipients are reviewed in Seidel, “Critical review of embryo transfer procedures with cattle” IN FERTILIZATION AND EMBRYONIC DEVELOPMENT IN VITRO (L Mastroianni, Jr. et al., eds., Plenum Press, New York, N.Y., 1981), the contents of which are hereby incorporated by reference.

The present invention can also be used to clone genetically engineered or transgenic animals. As explained above, the present invention is advantageous in that transgenic procedures can be simplified by working with a somatic cell source that can be clonally propagated. In particular, the somatic cells used for donor nuclei, which may or may not be serum-starved, have a desired DNA sequence inserted, removed or modified. Those genetically altered, somatic cells are then used for nuclear transplantation with enucleated oocytes.

Any known method for inserting, deleting or modifying a desired DNA sequence from a mammalian cell may be used for altering the somatic cell to be used as the nuclear donor. These procedures may remove all or part of a DNA sequence, and the DNA sequence may be heterologous. Included is the technique of homologous recombination, which allows the insertion, deletion or modification of a DNA sequence or sequences at a specific site or sites in the cell genome. A preferred method is the positive/negative selection method patented by Capecchi (U.S. Pat. No. 5,631,153, 5,627,059, and 5,847,982) or vectors reported in U.S. Pat. No. 6,110,735, 5,948,653, 5,925,577, 5,830,698, 5,776,777, 5,763,290, 5,574,205, and 5,527,644, all of which are incorporated by reference in their entirety.

The present invention can thus be used to provide adult animals, such as cows, with desired genotypes. Multiplication of adult animals with proven genetic superiority or other desirable traits is particularly useful, including transgenic or genetically engineered animals, and chimeric animals. Thus, the present invention will allow production of single sex offspring, and production of animals having improved meat production, reproductive traits and disease resistance. Furthermore, cell and tissues from the NT fetus, including transgenic and/or chimeric fetuses, can be used in cell, tissue and organ transplantation for the treatment of numerous diseases, as described below, in connection with the use of CICM cells. Hence, transgenic animals have uses including models for diseases, xenotransplantation of cells and organs, and production of pharmaceutical proteins.

For production of CICM cells and cell lines, the activated NT units are cultured under conditions which promote cell division without differentiation to provide for cultured NT units. After cultured NT units of the desired size are obtained, the cells are mechanically removed from the zona pellucida and are then used. This is preferably effected by taking the clump of cells which comprise the cultured NT unit, which typically will contain at least about 50 cells, washing such cells, and plating the cells onto a feeder layer, e.g., irradiated fibroblast cells. Typically, the cells used to obtain the stem cells or cell colonies will be obtained from the inner most portion of the cultured NT unit which is preferably at least 50 cells in size. However, cultured NT units of smaller or greater cell numbers as well as cells from other portions of the cultured NT unit may also be used to obtain ES cells and cell colonies. The cells are maintained on the feeder layer in a suitable growth medium, e.g., alpha MEM supplemented with 10% FCS and 0.1 mM β-mercaptoethanol (Sigma) and L-glutamine. The growth medium is changed as often as necessary to optimize growth, e.g., about every 2-3 days.

This culturing process results in the formation of CICM cells or cell lines. One skilled in the art can vary the culturing conditions as desired to optimize growth of the particular CICM cells. Also, for example, genetically engineered or transgenic cow CICM cells may be produced according to the present invention. That is, the methods described above can be used to produce NT units in which a desired DNA sequence or sequences have been introduced, or from which all or part of an endogenous DNA sequence or sequences have been removed or modified. Those genetically engineered or transgenic NT units can then be used to produce genetically engineered or transgenic CICM cells.

The resultant CICM cells and cell lines have numerous therapeutic and diagnostic applications. Most especially, such CICM cells may be used for cell transplantation therapies.

In this regard, it is known that mouse embryonic stem (ES) cells are capable of differentiating into almost any cell type, e.g., hematopoietic stem cells. Therefore, the CICM cells produced according to the invention should possess similar differentiation capacity. The CICM cells according to the invention will be induced to differentiate to obtain the desired cell types according to known methods. For example, the subject cow CICM cells may be induced to differentiate into hematopoietic stem cells, neural cells, muscle cells, cardiac muscle cells, liver cells, cartilage cells, epithelial cells, urinary tract cells, neural cells, etc., by culturing such cells in differentiation medium and under conditions which provide for cell differentiation. Medium and methods which result in the differentiation of CICM cells are known in the art as are suitable culturing conditions.

For example, Palacios, et al., Proc. Natl. Acad. Sci. USA 92: 7530-7 (1995) teaches the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferral of cell aggregates to a substrate which provides for cell attachment.

Moreover, Pedersen, J. Reprod. Fertil. Dev. 6: 543-552 (1994), a review article, references numerous articles disclosing methods for in vitro differentiation of embryonic stem cells to produce various differentiated cell types including hematopoietic cells, muscle, cardiac muscle, nerve cells, among others. These references and in particular the disclosures therein relating., to methods for differentiating embryonic stem cells are incorporated by reference in their entirety herein.

Thus, using known methods and culture mediums, one skilled in the art may culture the subject somatic cells and cells created using somatic cell nuclei to obtain cells for producing transgenic or chimeric animals.

The present invention has been described with reference to a preferred embodiment. However, it will be readily apparent to those skilled in the art that it is possible to embody the invention in specific forms other than as described above without departing from the spirit of the invention. The embodiments described in the examples below are illustrative and should not be considered restrictive in any way. The scope of the invention is given by the appended claims, rather than the preceding description, and all variations and equivalents which fall within the range of the claims are intended to be embraced therein.

### EXAMPLE 1

### Effect of Confluency and Cell Age on Cell Cycle Length

Establishment offetal cell lines. Bovine fetuses were obtained from a slaughterhouse and the crown-rump length was measured. After washing in rinse solution (DPBS containing antibiotic/antimycotic (Sigma) and Fungizone (Gibco) and removing the head and internal organs, the remaining tissues were finely chopped into pieces, using scalpel blades. Tissue pieces were washed twice in rinse solution by allowing the pieces to settle to the bottom of 50 ml tubes and removing the supernatant. To the tissue pieces, 30-40 ml of 0.08% trypsin (Difco) and 0.02% EDTA (Sigma) in PBS (Gibco) was added, and the tissue incubated for 30 min. at 39° C., 5% CO₂. At intervals of 30 mm., the supernatant was carefully removed and centrifuged in another tube for 5 mm. at 300×g. Then the tissue pieces were separated by removing the supematant, adding another 30-40 ml of 0.08% trypsin and 0.02% EDTA in PBS, and incubating the tissue samples again for 30 min. at 39° C., at 5% CO₂. The supematant then carefully was removed leaving the tissue pieces in a 50 ml tube; an equal volume of alpha MEM (Gibco) supplemented with 10% FCS (fetal calf serum, Hyclone), glutamine (Sigma), mercaptoethanol (Gibco) and antibiotic/antimycotic was added to the tissue, and the tissue was centrifuged at 1,000×g for 5 min. The pellet was carefully separated by aspirating off the supernatant. The tissue was resuspended in alpha MEM supplemented with the above components and seeded on 100 mm tissue culture plates (Coming) and incubated at 39° C., 5% CO₂. The tissue pieces were again incubated with the trypsin-EDTA in PBS solution, the supernatant collected, and the cells seeded as described above. On day three of seeding, the cells were harvested, using trypsin-EDTA solution and counted. One million cells were selected and re-seeded in 100 mm tissue culture plates, and the remaining cells were frozen in alpha MEM with 10% DMSO (Sigma). Other adherent similarly cells can be prepared, as would be known by the skilled artisan.

Establishment of calf and adult cell lines. Ear punches were taken (1 mm) after thoroughly cleaning the skin surface by clipping the hair and washing with disinfectant. The ear punch samples were washed three times in rinse solution, and the cartilage portion was separated removed out in between the outer and inner surface of the skin. Samples were explanted in 100 mm tissue culture plates and covered with a glass slide in order to prevent floating in the culture media. After making the explant, 10 ml of alpha MEM supplemented with the components that were used in the establishment of fetal cell lines (above), was added and incubated at 39° C., 5% CO₂. After removal of the explants on day 10, monolayers of cells were harvested using 0.08% trypsin and 0.02% EDTA in PBS solution, counted and re-seeded in 100 mm tissue culture plates.

Population doublings and cell counts. After the initial seeding, cells were counted when they were 90% confluent by standard trypsinization procedure using 20 0.08% trypsin and 0.02% EDTA in PBS solution. Harvested cells were centrifuged at 1000×g for 5 min., and the cell pellet was resuspended in 10 ml of alpha MEM. A suitable sample of cells was counted using hemocytometer. When these cultures were 95% confluent, they were harvested, counted and the population doublings were calculated. This procedure was repeated until these cells reach senescence. Excess cells obtained during the harvesting and re-seeding steps were frozen in supplemented alpha MEM and 10% DMSO (Sigma) and stored in liquid nitrogen.

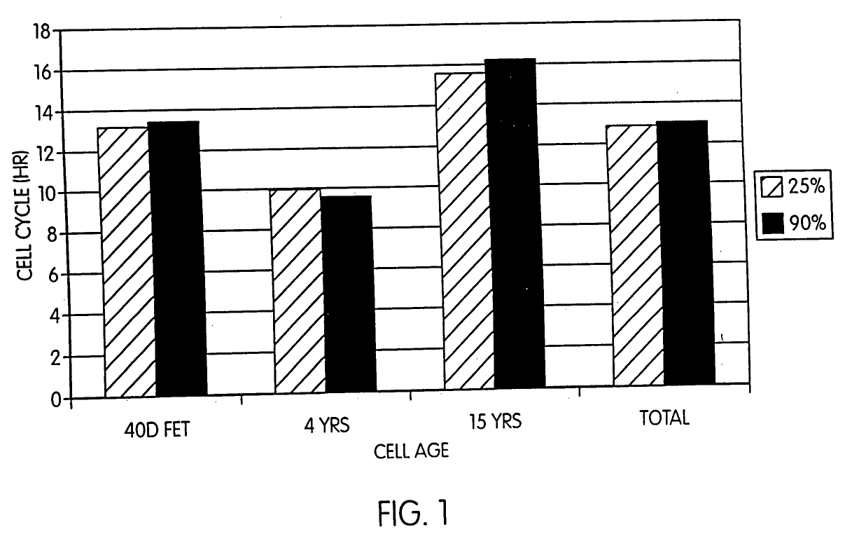

Cell fixation, staining andflow cytometry. Cell cycle comparisons were made between cells from different confluencies (FIG. 1). After over night fixation of the cells in 70% ethanol, cells were washed thoroughly with chilled PBS and treated with 10 RNase followed by incubation for 2-3 hrs at 37° C. After incubation, cells were stained with propedium iodide (Sigma).

Isolation of dividing G₁ cells. 24-hours prior to “shake-off,” 5.0×10⁵ cells were plated onto 100 mm Corning tissue culture plates containing 10 ml of alpha MEM supplemented with 10% FCS. The following day, plates were washed with PBS, and culture medium was replaced about 1 to about 2 hours before shake-off. These plates were vortexed for 30-60 seconds, the medium removed and centrifuged, and the cell pellet resuspended in 250 μ1 of culture medium.

Cells attached by a cytoplasmic bridge have just under gone cytokinesis and are in early G₁. In the following experiments, these early G₁ cells were identified (by visual inspection) based on this characteristic and used.

Bdru labeling of G₁ doublets. G₁ doublet cells were placed in Lab-Tek 4-well culture chambers (Nunc) containing 250 μ1 of alpha MEM supplemented with bromodeoxyuridine (Bdru)(Boehringer Mannheim). At 0, 2, 4, and 7 hours, cells were fixed with 70% ethanol (in 50 mM glycine buffer, pH 2.0) for about 20 minutes. Following fixation, cells were washed and incubated with anti-Bdru for 30 min. at 37° C. After 30 min., cells were washed and anti-mouse-Ig-fluorescein was added; fixed cells were then incubated again for an additional 30 minutes at 37° C. Following the second incubation, fixed cells were washed and mounted with glycerol. The percent of cells in S-phase was determined using an epi-fluororescent microscope (Nikon).

Assessment of Cell-Cycle Length. G₁ doublets were isolated via standard micromanipulation using a 25 μm beveled needle. Individual doublets were transferred into 50 A⁴ drops of alpha MEM supplemented with 10% FCS derived from actively dividing cultures of fibroblasts (conditioned medium). The time of pick-off was marked 0 hours, and every 2 hours there after isolated doublets were assessed for cell division. Ten microdrops per culture plate were assessed, and the proportion of cells that divided within 24 hours were used to calculate the mean cell-cycle length.

The results for FIG. 1 were obtained by measuring the cell cycle length of cells at 90% confluency versus cell cycle length at 25% confluency. Cells were obtained and plated as described above. For the most part, cell cycle length was less for cells grown at 25% confluency rather than at 90% confluency.

### EXAMPLE 2

### Effect of Time in Culture and Donor Age on Cell Growth Rate

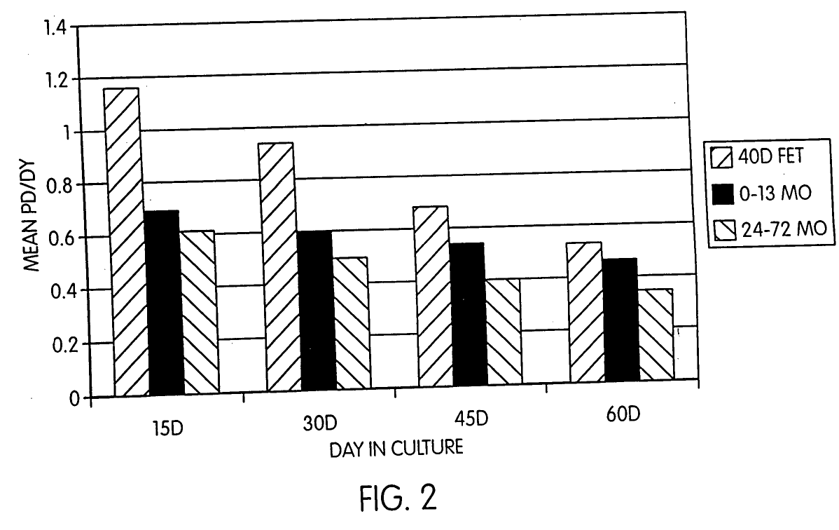

FIG. 2 demonstrates that increased time in culture for the cells obtained as described above, leads to a decrease in population divisions or doubling per day (PD/DY).

### EXAMPLE 3

### Length of G₁ in Fibroblasts Recovered from Culture

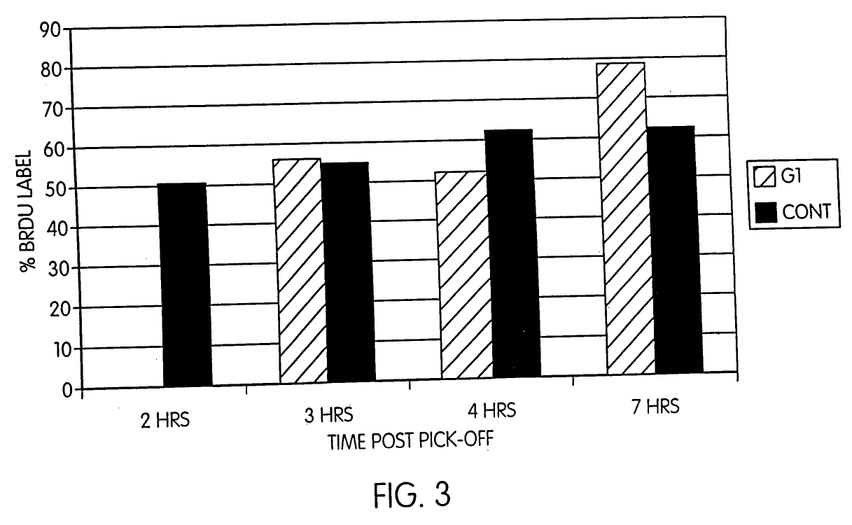

Fibroblast cells were obtained, cultured and harvested as described in Example 1. FIG. 3 shows the length of G₁ in fibroblasts recovered from culture after pick-off.

### EXAMPLE 4

### Preparing Somatic Cells for Nuclear Transfer Using Cell Shake-Off and Mitotic Doublet Cell Selection at Low Confluency.

Cells are prepared for nuclear transplantation as described above in Example 1.

Nuclear transplantation. In vitro matured oocytes were stripped with 1.0% hyaluronidase at 18 hpm. Oocytes were briefly washed in TL-hepes and then stained with Hoescht 33342 (Sigma) for 20 min. The cells were enucleated using a 18-20 μm beveled needle. Enucleation was confirmed with UV light. The donor cell was transferred using a 20 μm needle and fused in a sorbitol based medium with one electrical pulse of 115 mV for 20 sec (Electrocell manipulator 200, San Diego, Calif.) at 24 hours.

Activation. At 28 hpm, reconstructed oocytes and controls were chemically activated using a Ca ionophore (5 mM) for 4 min. (Cal Biochem) and DMAP (200 mm) for 3.5 hours. At 3.5 hours post activation, oocytes were briefly washed in HCEM hepes and transferred into culture.

In vitro culture of nuclear transfer embryos. Embryo culture was performed in 4-well tissue culture plates (Nunc), containing mouse blocked feeder layer and 0.5 ml of culture media covered with 200 μ1 of embryo tested mineral oil (Sigma). 25-50 embryos were placed in each well and incubated at 39° C., 5% CO₂. On day four, 10% FCS was added to the culture media. On days 7 and 8, develoment to blastocyst was 10 recorded. Cell numbers were described by mounting the cells with 1% Hoechst in glyceral (Sigma).

The donor somatic cell utilized preferably is any type of adherent cell. Other similar methods and materials may be substituted and used as would be known to the skilled artisan.

### EXAMPLE 5

### Preparing Somatic Cells for Nuclear Transfer Using Cell Shake-Off and Mitotic Doublet Cell Selection at Low Confluency and Extending G₁ Phase Using a Cooling Step.

The G₁ phase of the somatic cell can be extended by placing the cells at 4° C. and performing the steps for nuclear transfer, as described above.

### EXAMPLE 6

### Preparing Somatic Donor Cells for Nuclear Transplantation Using Mitotic Shake-Off and Doublet Cell Selection in Combination with Serum Starvation.

The methods and materials used above can also be utilized in combination with agents that synchronize cells in G₁, such as certain kinase inhibitors (e.g., KT5720, KT5823 or KT5926). Cells can be obtained via shake-off as described above. Cells can then be resuspended in media containing a kinase inhibitor in any one of the following concentrations: KT5720 at about 11, μM, KT5823 at about 15 μM, KT5926 at about 3 μM or K252b at about 11 μM. G₁ phase can be increased further by placing the cells at 4° C. if G₁ phase is to be further lengthened. The cells can then be utilized as previously described.

All references are herein incorporated by reference in their entirety.

1. A method of selecting and using donor somatic cells for nuclear transfer or nuclear transplantation comprising the steps of:
(A) synchronizing cell cycle of donor somatic cells by mechanically dislodging the cells from the surface of the culture dish;
(B) selecting somatic doublet cells; and
(C) using said selected cells or the nuclei of said somatic doublet cell in nuclear transfer or nuclear transplantation.

2. A method of preparing donor somatic cells for nuclear transfer or nuclear transplantation comprising the steps of:
(A) obtaining cells which are about 25% to about 50% confluent and plating the cells about 24 hours to the synchronization step;
(B) synchronizing cell cycle of donor somatic cells by mechanically dislodging the cells from the surface of the culture dish;
(C) selecting somatic doublet cells; and
(D) using the nuclei of the somatic doublet cell in nuclear transfer or nuclear transplantation.

3. A method of preparing donor somatic cells for nuclear transfer or nuclear transplantation comprising the steps of:
(A) obtaining confluent cells and plating the cells about 24 hours to the synchronization step;
(B) synchronizing cell cycle of donor somatic cells by mechanically dislodging the cells from the surface of the culture dish;
(C) selecting somatic doublet cells; and
(D) using the nuclei of the somatic doublet cell in nuclear transfer or nuclear transplantation.

4. The method of claim 1, further comprising the step of cooling the selected mitotic doublet cells to extend their G₁ phase.

5. The method of claim 4, wherein the cells are cooled to 4° C.

6. The method of claim 1, wherein the selected cells are then cultured in media lacking at least one of the following: serum, isoleucine, glutamine or phosphate.

7. The method of claim 1, wherein a G₁ synchronizing agent is added to the media of the selected cells to lengthen their G₁ phase.

8. The method of claim 7, wherein the G₁ synchronizing agent is selected from the group consisting of: aphidicolin, mimosine, KT5823, KT5720, KT5926 and K252b.

9. The method of claim 1, wherein the cells are mechanically dislodged when the cells are about 20% to about 50% confluent.

10. The method of claim 9, wherein the cells are shaken when the cells are about 25% confluent.

11. The method of claim 1, wherein the cells are mechanically dislodged when the cells are confluent.

12. A method of preparing a transgenic animal comprising the steps of:
(A) preparing donor somatic cells according to claim 1;
(B) isolating nuclei from said selected somatic cells;
(C) inserting the nuclei into at least one enucleated embryonic stem (ES) cell, embryonic germ (EG) cell, enucleated embryo, or enucleated somatic cell under conditions suitable for the formation of a nuclear transfer (NT) unit to yield a fused NT;
(D) activating said fused NT unit to yield an activated NT unit; and
(E) transferring said activated NT unit to a host mammal such that the activated NT unit develops into a fetus.

13. A method of preparing a transgenic animal comprising the steps of:
(A) preparing somatic cells according to claim 1;
(B) isolating nuclei from said selected somatic cells;
(C) inserting the nuclei into either an enucleated oocyte, enucleated sperm, enucleated embryo, or enucleated somatic cell under conditions suitable for the formation of a nuclear transfer (NT) unit to yield a fused NT unit;
(D) activating said fused NT unit to yield an activated NT unit; and
(E) transferring said activated NT unit to a host mammal such that the activated NT unit develops into a fetus.

14. A method of preparing a chimeric animal comprising the steps of:
(A) preparing somatic cells according to claim 1;
(B) isolating nuclei from said selected somatic cells;
(C) inserting the nuclei into at least one enucleated ES cell or enucleated EG cell under conditions suitable for the formation of a nuclear transfer (NT) unit to yield a fused NT unit;
(D) activating said fused NT unit to yield an activated NT unit; and
(E) inserting said activated NT unit in to a host mammal embryo such that the embryo develops into a fetus.
